(19) United States
(12) Patent Application Publication
(10) Pub. No.: US 2006/0259017 A1
(43) Pub. Date: Nov. 16, 2006

(54) Adhesive elements and methods for accessing the pericardial space

(75) Inventors: Ronald W. Heil JR., Roseville, MN (US); Randy W. Westlund, River Falls, WI (US); Peter L. Callas, Castro Valley, CA (US)

Correspondence Address: GUIDANT PATENT DOCKETING;FAEGRE & BENSON, LLP, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/115,647

(22) Filed: Apr. 27, 2005

(51) Int. Cl. A61B 17/00

(52) U.S. Cl. 606/1; 606/228; 600/201; 606/213

(57) ABSTRACT

A technique for accessing a pericardial space of a heart includes attaching a grip to a pericardium with adhesive, grasping the grip, drawing the pericardium away from the heart, and perforating the pericardium. The grip may consist solely of an adhesive. In other embodiments, other structures for the grip are used, including anchors, suture loops, and gripping tubes.

### TECHNICAL FIELD

The present invention relates to medical devices and methods for accessing an anatomical space of the body. More specifically, the invention relates to devices and methods for accessing the pericardial space of the heart in a minimally-invasive manner.

### BACKGROUND

The human heart is enveloped within a tissue structure referred to as the pericardium, which comprises two major parts. The inner layer of the pericardium lies immediately over the myocardium (heart muscle) and is referred to as the visceral pericardium or epicardium. The outer layer, forming a sac around the visceral pericardium, is referred to as the parietal pericardium. Normally these two layers lie in close contact with each other and are separated only by a thin layer of pericardial fluid, which allows the heart to move within the sac with minimal friction. The potential space between the visceral and parietal pericardia is referred to as the pericardial space. The visceral pericardium is typically referred to as the epicardium and the parietal pericardium is typically referred to as the pericardium. This naming convention will be used hereafter.

Access to the pericardial space is necessary for a variety of medical procedures, including treatment of infections, injuries and heart defects. For example, cardiac rhythm management systems such as pacemakers, implantable pulse generators, and implantable cardioverter defibrillators include leads having electrodes for sensing and stimulating the heart. These leads can be deployed inside or outside the heart. In the latter case, the pericardial space must be traversed to reach the epicardium or myocardium for lead implantation and attachment.

Part of the challenge in accessing the pericardial space stems from its minimal thickness. When making an incision or perforation in the pericardium, it is preferable to avoid also puncturing the underlying epicardium and damaging the myocardium or a coronary vessel. However, the close proximity of the epicardium to the pericardium makes this difficult. Another important consideration is the widespread trend toward minimally-invasive surgical techniques, which generally offer a host of advantages including lower costs and fewer complications.

Other known methods of accessing the pericardial space include (a) use of an endoscopic grasper and scissors or (b) careful needle placement under fluoroscopic vision. The grasper and scissors approach requires multiple surgical access ports, while the needle technique has resulted in many reported incidents of myocardial puncture, some requiring emergency surgery. Various exemplary methods for accessing this space are disclosed in U.S. Pat. Nos. 4,865,037 and 5,033,477 to Chin et al.

There exists a need for improved, efficacious methods and instruments for penetrating the pericardium and thereby accessing the pericardial space that minimize the risk of damaging other tissues of the heart. There is a further need for such methods that are compatible with minimally-invasive surgical techniques would be especially desirable.

### SUMMARY

In one embodiment, the present invention is a method for accessing a pericardial space located between a pericardium and an epicardium. An adhesive grip is attached to a surface of the pericardium. The adhesive grip is grasped. The adhesive grip is drawn away from the epicardium, thereby enlarging the pericardial space. The pericardium is perforated with a perforation implement. In one embodiment, the adhesive grip further comprises an adhesive and attaching the adhesive grip includes curing the adhesive. According to another embodiment, the adhesive grip further comprises an adhesive and a gripping element.

In another embodiment, the present invention is a method for accessing a pericardial space located between a pericardium and an epicardium. A first end of a gripping tube is attached to a surface of the pericardium with an adhesive. The gripping tube is withdrawn from the epicardium, thereby enlarging the pericardial spaced. The pericardium is perforated with a perforation element. According to one embodiment, attaching the first end of the gripping tube includes applying adhesive to at least one of the first end of the gripping tube and the surface of the pericardium, contacting the first end of the gripping tube with the surface of the pericardium and curing the adhesive. According to another embodiment, the first end of the gripping tube includes a detachable tip.

According to yet another embodiment, the present invention is a pericardial access system for facilitating access to a pericardial space between a pericardium and an epicardium. The system includes a tubular body and a perforation instrument for perforating the pericardium. The tubular body defines an inner lumen adapted for slidably receiving a medical device therethrough and has a distal surface adapted for adhesively coupling to the pericardium. The perforation instrument is adapted to slide within the inner lumen and extend beyond the distal surface.

According to still another embodiment, the present invention is a pericardial grip that can be grasped for purposes of manipulating a pericardium. The pericardial grip includes a graspable element and an adhesion means for facilitating adhesion of the graspable element to an exterior surface of the pericardium.

This summary is not intended to describe each embodiment or every implementation of the present invention. Advantages and a more complete understanding of the invention will become apparent upon review of the detailed description and claims in conjunction with the accompanying drawings.

### BRIEF DESCRIPTION OF THE DRAWINGS

FIG. 1 is an illustration of a heart having an adhesive grip attached to the pericardium in accordance with a first embodiment of the present invention.

FIG. 2 is a partially-sectioned side view of the adhesive grip as shown in FIG. 1 along with associated surgical tools in relation to the underlying anatomic layers of the heart.

FIG. 3 is a flowchart showing a method of accessing a pericardial space using the adhesive grip as shown in FIG. 2.

FIG. 4 is a partially-sectioned side view of the adhesive grip as shown in FIG. 2 with a pair of forceps engaging the adhesive grip such that the pericardium drawn away from the heart.

FIG. 5 is a partially-sectioned side view of the adhesive grip, pericardium, and forceps as shown in FIG. 4, with a perforation implement perforating the pericardium.

FIG. 6 is a partially-sectioned side view of the adhesive grip, forceps, and perforation implement as shown in FIG. 5, with a guide wire advanced into the pericardial space.

FIG. 7 is a partially-sectioned side view of an adhesive grip including a suture loop and associated surgical tools in relation to the underlying anatomic layers of the heart in accordance with a second embodiment of the present invention.

FIG. 8 is a flowchart detailing a method of accessing a pericardial space using the adhesive grip as shown in FIG. 7.

FIG. 9 is a partially-sectioned side view of an adhesive grip including a suture loop with an anchor with associated surgical tools in relation to the underlying anatomic layers of the heart in accordance with a third embodiment of the present invention.

FIG. 10 is a flowchart detailing a method of accessing a pericardial space using the adhesive grip as shown in FIG. 9.

FIG. 11 is a perspective view of an adhesive grip in accordance with another embodiment of the present invention.

FIG. 12 is a flowchart detailing a method of accessing a pericardial space using the adhesive grip as shown in FIG. 11.

FIG. 13 is a partially-sectioned side view showing the adhesive grip of FIG. 11 attached to a pericardium, with the underlying anatomic layers of the heart.

FIG. 14 is a partially-sectioned side view of the adhesive grip and anatomic layers of the heart as shown in FIG. 13, with the pericardium drawn away from the heart.

FIG. 15 is a partially-sectional side view of the adhesive grip and anatomic layers of the heart as shown in FIG. 14, with a slidably mounted perforation implement perforating the pericardium.

FIG. 16 is a side perspective view of an adhesive grip in accordance with another embodiment of the present invention.

FIG. 17 is a side perspective view of the adhesive grip of FIG. 16 and further including a plurality of flexible tabs at a distal end.

FIG. 18 is a side perspective view of the adhesive grip of FIG. 17 mounted on the pericardium.

FIG. 19 is a side sectional view of an adhesive grip including a detachable inner tip in accordance with another embodiment of the present invention.

FIG. 20 is a side sectional view of an adhesive grip including an detachable outer tip in accordance with one embodiment of the present invention.

FIG. 21A is a side perspective view of an adhesive grip including a plurality of adhesive lumens in accordance with another embodiment of the present invention.

FIG. 21B is a cross-sectional view of the adhesive grip of FIG. 21A taken along line B-B.

FIG. 21C is a cross-sectional view of the adhesive grip of FIG. 21A taken along line C-C.

FIG. 22 is a flowchart detailing a method of accessing a pericardial space with the adhesive grip of FIGS. 21A-21C.

FIG. 23 is a partially-sectioned view of the adhesive grip of FIGS. 21A-21C in relation to the anatomic layers of the heart.

FIG. 24 is a partially-sectioned view of the adhesive grip of FIG. 23 adhered to the pericardium.

FIG. 25 is a partially-sectioned view of the adhesive grip of FIGS. 23 and 24 including a perforation implement.

FIG. 26 is a partially-sectioned view of an adhesive grip and a perforation instrument disposed within a sleeve.

FIG. 27 is a partially-sectioned view of the adhesive grip of FIG. 26 in which the perforation instrument is removed.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

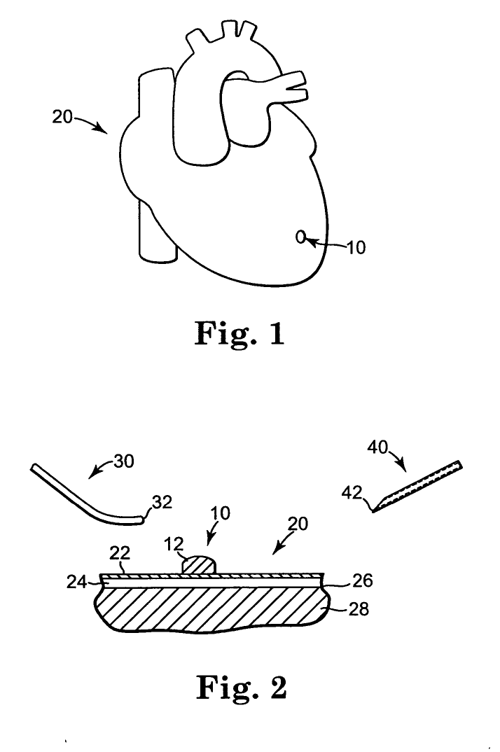

FIG. 1 is a general schematic illustration of an adhesive grip 10, in accordance with a first embodiment of the present invention, in relation to a heart 20. As shown in greater detail in the partially-sectioned side view of FIG. 2, the adhesive grip 10 includes a bolus 12 of bio-compatible adhesive that has been deposited on a pericardium 22 of the heart 20. In the illustrated embodiment, the bolus 12 solely or substantially forms the grip 10. However, other materials and elements can be used in adhesive grips in other embodiments such as those described below. A pericardial space 24 lies opposite the pericardium 22 and is bounded by an epicardium 26 on its far side. The epicardium 26 contacts a myocardium 28 on the side opposite the pericardial space 24. The upper left portion of FIG. 1 shows a pair of forceps 30 having a tip 32 for grasping the adhesive grip 10. The upper right portion of FIG. 1 shows a perforation implement 40 with a hollow sharpened element 42.

The bolus 12 may be formed using a biocompatible adhesive formed of a high viscosity gel. The bio-compatible adhesive used to form bolus 12 can be characterized by a high viscosity so that it will maintain a form after deposition. Non-limiting examples of adhesives that can be used for this purpose include BioGlue® available from CryoLife, Inc. of Kennesaw, Ga.), fibrin sealants, cyanoacrylates, bovine collagen/thrombin, and polyethylene glycol polymers. Degradable or absorbable bio-compatible adhesives may be used, thereby leaving no permanent residual. Alternately, any permanent residual of a bio-compatible adhesive may be naturally encapsulated and rendered inert. In addition, drug-eluting adhesives can be employed to deliver a therapeutic benefit to the heart. Such adhesives may be cured or hardened via exposure to the environment, under the application of light, RF, or through the addition of a curing agent. An appropriate adhesive might be one that cures at body temperatures. Another appropriate adhesive might be one that is thermo-set. Another appropriate light curing adhesive is the acrylic adhesive Loctite® 3321 (available from Loctite® of Rocky Hill, Conn.). Other adhesives may cure through the application of particular radio frequency or may

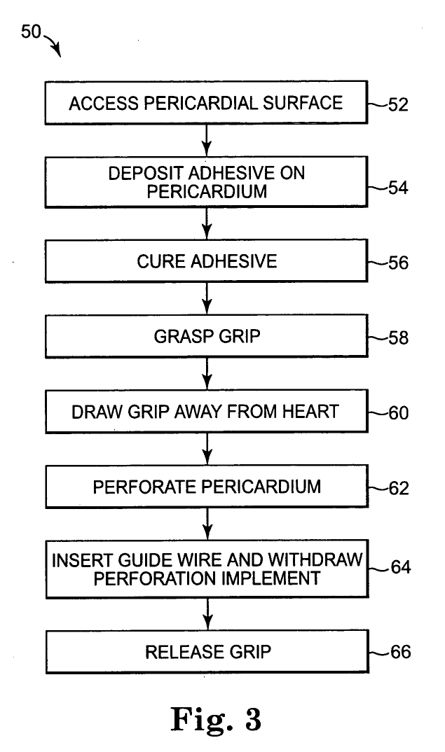

The flowchart of FIG. 3 shows one embodiment of a method 50 for accessing the pericardial space using the adhesive grip 10 shown in FIG. 2. As shown in FIG. 3, a minimally-invasive surgical technique is used to gain access to the pericardium 22 (block 52). Any appropriate conventional or otherwise known technique can be used for this purpose. An exemplary pericardial access technique is described in published U.S. patent application Ser. No. 10/140,309 to Chin et al., which is herein incorporated by reference. The adhesive grip 10 is attached to the accessed pericardium 22. This includes the steps of depositing the bolus 12 of adhesive on the pericardium 22 (block 54) and curing or otherwise hardening the bolus 12 of adhesive (block 56). Any efficacious deposition technique can be used here, such as application of the adhesive with a swab or paddle, or extrusion of the adhesive from a tubular applicator. The length of curing time will depend on the type of adhesive used and may depend on factors such as, for example, the addition of a curing agent, the presence of moisture, illumination with light, or application of pressure.

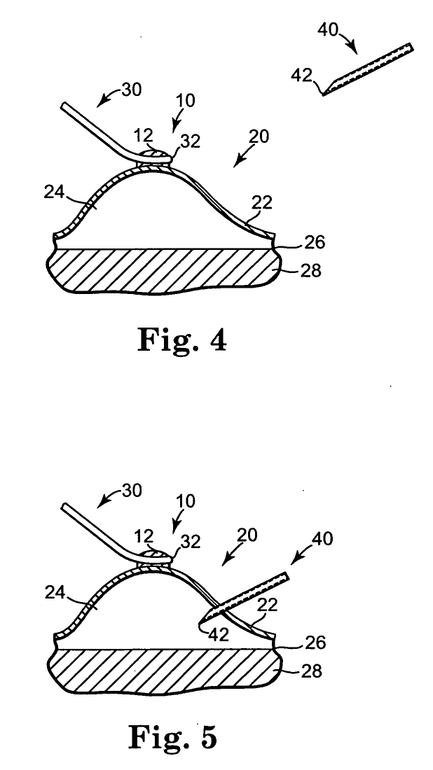

As shown in FIG. 4, after the bolus 12 of adhesive has cured, the forceps 30 or another suitable instrument is used to grasp the adhesive grip 10 (block 58) and the grip is drawn away from the heart 20 to enlarge the pericardial space 24 (block 60). With the pericardium 22 withdrawn, a perforation implement 40 is advanced through the pericardium 22 to perforate it (block 62) as illustrated in FIG. 5. Perforating the pericardium 22 while the pericardium 22 is drawn away from its nominal position in close proximity to underlying heart tissues allows the perforation to be performed with low risk of puncturing or otherwise damaging other tissues.

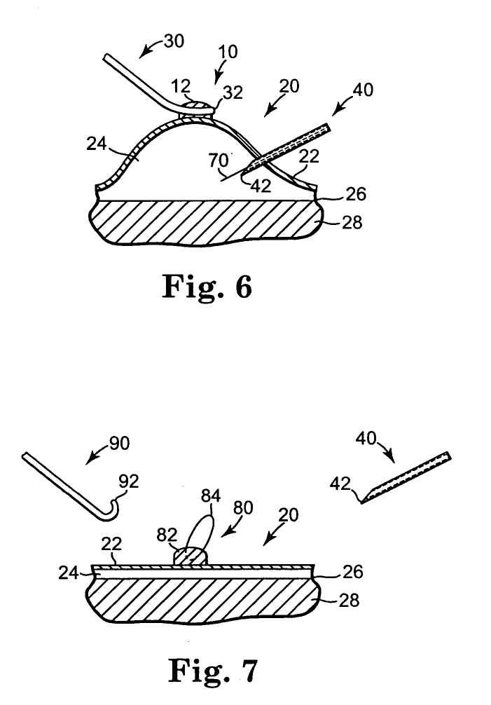

A guide wire 70 can then be passed through a lumen in the implement 40 and the implement withdrawn (block 64), as shown in FIG. 6. The forceps 30 can then be returned toward the heart 20 and the grasp on the adhesive grip 10 released (block 66).

FIG. 7 is a partially-sectioned side view of an adhesive grip 80 in accordance with another embodiment of the present invention. In this figure, the anatomy is identical to that of FIG. 2, including a pericardium 22, pericardial space 24, epicardium 26, and myocardium 28 of a heart 20. In the present embodiment, the adhesive grip 80 includes a loop 84 of bio-compatible material, such as is known in the art of sutures, embedded in a bolus 82 of bio-compatible adhesive that has been deposited on the pericardium 22. The adhesive used for bolus 82 can be the same as or similar to those used in connection with grip 10 described above. The upper left portion of FIG. 7 shows a surgical tool 90 with a hook-shaped distal end 92 that can be used to grasp the suture loop 84. The upper right portion of FIG. 7 shows a perforation implement.

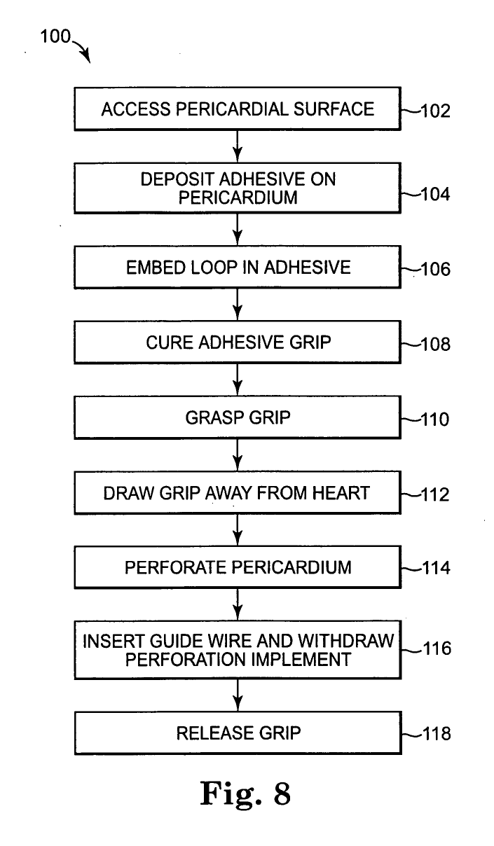

The flowchart of FIG. 8 shows a method 100 for accessing the pericardial space 24 using the adhesive grip 80 shown in FIG. 7. A minimally-invasive surgical technique is used to gain access to the pericardium 22 (block 102). The adhesive grip 80 is attached to the accessed pericardium 22. This includes the steps of depositing the bolus 82 of adhesive on the pericardium 22 (block 104), embedding the suture loop 84 in the bolus 82 (block 106), and curing the adhesive of the bolus 82 (block 108). After the bolus 82 of adhesive has cured, the hooked surgical tool 90 or another suitable instrument is used to grasp the adhesive grip 80 by hooking loop 84 with hooked distal end 92 of surgical tool 90 (block 110) and the grip 80 is drawn away from the heart 20 to enlarge the pericardial space 24 (block 112). With the pericardium 22 withdrawn, a perforation implement 40 is advanced through the pericardium 22 to perforate it (block 114). A guide wire can then be passed through the lumen in the implement 40 and the implement 40 withdrawn (block 116). The hooked tool 90 can then be returned toward the heart 20 and the adhesive grip 80 released (block 118).

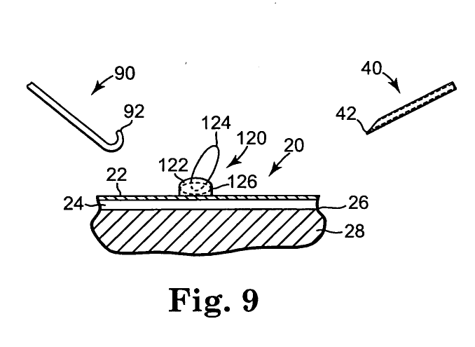

FIG. 9 is a partially-sectioned side view of an adhesive grip 120 in accordance with a third embodiment of the present invention. In FIG. 9, the anatomy is identical to that of FIGS. 2 and 7, including a pericardium 22, pericardial space 24, epicardium 26, and myocardium 28 of a heart 20. In the present embodiment, the adhesive grip 120 includes a suture loop 124 attached to an anchor 126 embedded in a bolus 122 of adhesive that has been deposited on the pericardium 22. As in the other embodiments, all materials used are of a bio-compatible nature. The adhesive used for bolus 122 can be the same as or similar to those used in connection with grips 10 and 80 described above. For the anchor 126, possible materials include silicone, polyurethane and common moldable suture materials such as polyethylene, polypropylene, and polyvinylidene fluoride, as well as previously-cured adhesive of the same type used for the bolus 122. The anchor 126 provides increased surface are to prevent the loop 124 from pulling through the cured bolus 112 of adhesive, due to local repetitive stresses. The upper left portion of FIG. 9 shows a surgical tool 90 with a hook-shaped distal end 92 that can be used to grasp the suture loop 124. The upper right portion of FIG. 9 shows a perforation implement 40.

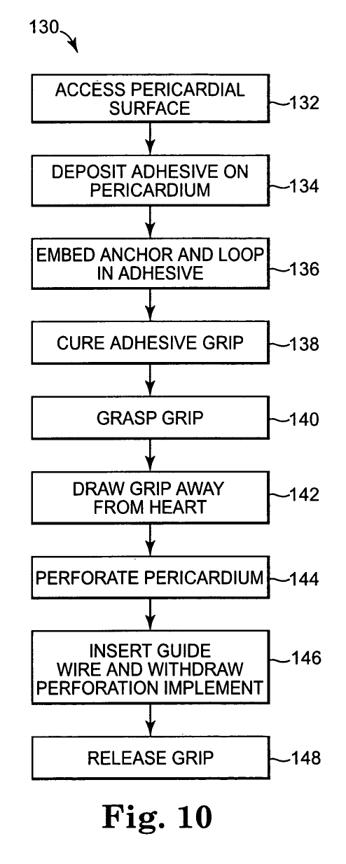

The flowchart of FIG. 10 shows a method 130 for accessing the pericardial space using the adhesive grip 120 shown in FIG. 9. A minimally-invasive surgical technique is used to gain access to the pericardium 22 (block 132). The adhesive grip 120 is attached to the accessed pericardium 22. This includes the steps of depositing the bolus 122 of adhesive on the pericardium 22 (block 134), embedding the anchor 126 attached to the suture loop 124 in the adhesive bolus 122 (block 136), and curing the adhesive of the bolus 122 (block 138). After the bolus 122 of adhesive has cured, the hooked surgical tool 90 or another suitable instrument is used to grasp the adhesive grip 120 (block 140) and the grip 120 is drawn away from the heart 20 to enlarge the pericardial space 24 (block 142). With the pericardium 22 withdrawn, a perforation implement 40 is advanced through the pericardium 22 to perforate it (block 144). A guide wire can then be passed through the lumen in the implement 40 and the implement withdrawn (block 146). The hooked tool 90 can then be returned toward the heart 20 and the grasp on the adhesive grip 120 released (block 148).

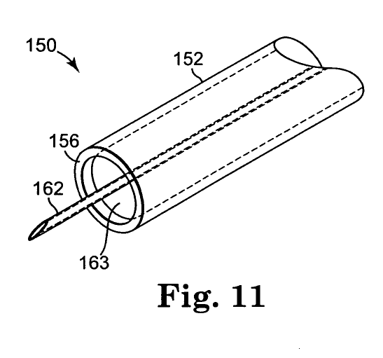

FIG. 11 is a perspective view of an adhesive grip 150 in accordance with another embodiment of the present invention. The grip 150 includes a substantially hollow tubular body 152. In one embodiment, a distal surface 156 of the tubular body 152 is textured so as to provide greater surface area for adhesion. A hollow, sharpened perforation implement 162 is mounted slidably within a central lumen 163 of the tubular body 152. Tubular body 152 has a substantially greater diameter and/or a greater wall thickness in comparison to the grips of the preceding embodiments so as to increase the surface area available for adhesion.

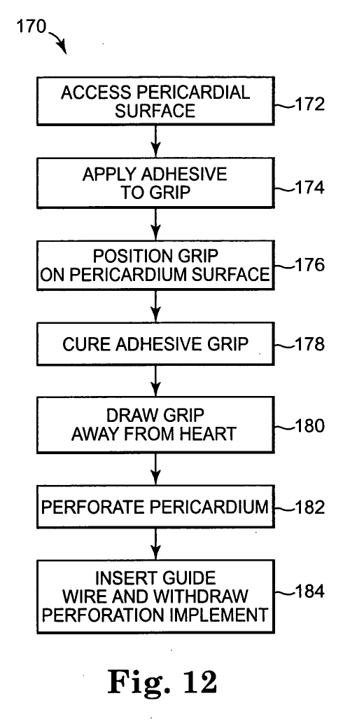

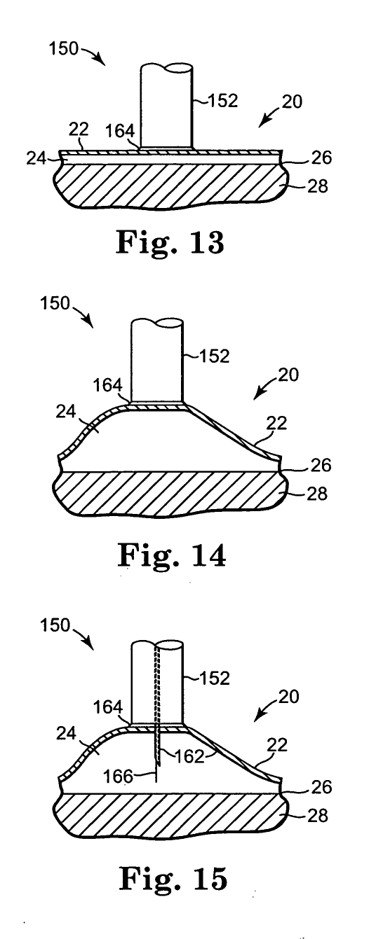

The flowchart of FIG. 12 shows a method 170 for accessing the pericardial space using the adhesive grip 150 shown in FIG. 11. A minimally-invasive surgical technique is used to gain access to a pericardium 22 (block 172). The grip 150 is attached adhesively to the pericardium 22 as depicted in FIG. 13. This includes the steps of applying an adhesive 164 to the distal surface 156 of the grip 150 (block 174), placing or contacting the grip 150 to an appropriate location on the pericardium 22 (block 176), and curing the adhesive 164 (block 178). After the adhesive 164 has cured, the grip 150 is drawn away from the heart 20 to enlarge the pericardial space 24 (block 180), as shown in FIG. 14. With the pericardium 22 withdrawn, the perforation implement 162 is advanced through the lumen 163 to perforate the pericardium 22 (block 182), as illustrated in FIG. 15. A guide wire 166 can then be passed through a lumen in the implement 162 into the pericardial space 24 and the implement 162 withdrawn (block 184).

Rather than applying adhesive 164 to the grip 150, a bolus of adhesive may be deposited on the pericardium 22, and the distal surface 156 of the grip 150 contacted with the adhesive on the percardium 22 before curing. Following the procedure, the adhesive bond is broken and the grip 150 is detached from the pericardium 22. A cutting tool may be used to detach the grip 150 from the adhesive 164.

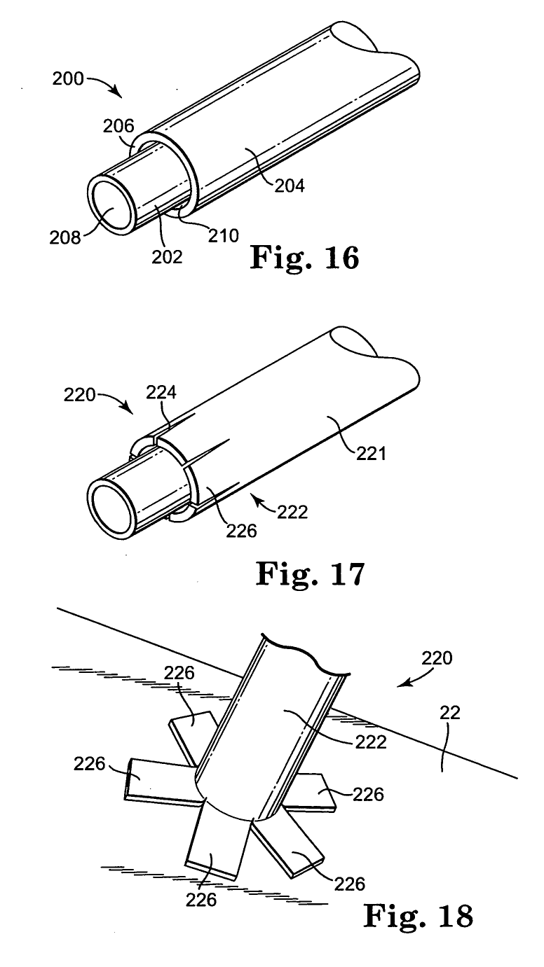

FIG. 16 is a perspective view showing yet another embodiment of an adhesive grip 200 similar to the adhesive grip 150 shown in FIGS. 11-15 but featuring a coaxial construction. In this embodiment, the grip 200 includes an inner substantially hollow tubular body 202 slidably disposed in an outer substantially hollow tubular body 204. In reference to method 170 described above and shown in FIG. 12, during step 174 the adhesive would be applied only to a distal surface 206 of the outer tubular body 204. In step 176, the inner tubular body 202 would be brought into contact with the pericardium 22 before the outer tubular body 204 would contact the pericardium 22. Applying the grip 200 in this manner would prevent adhesive from entering a lumen 208 of the inner tubular body 202 and potentially interfering with the perforation of the pericardium 22. Additionally, during the removal of the adhesive grip 200 from the pericardium 22 (block 184), the inner tubular body 202 may be used to stabilize the pericardium 22 as the outer tubular body 204 is manipulated for removal.

In still another embodiment, the adhesive grip 200 is used, but in reference to method 170 described above and shown in FIG. 12, during step 174, the adhesive is delivered to the distal surface 206 through an annular lumen 210 defined between the inner tubular body 202 and outer tubular body 204. The adhesive is injected or pumped along the annular lumen 210. After a small amount of adhesive has reached the distal end 206 of the outer tubular body 204, inner tubular body 202 and outer tubular body 204 are brought into contact with the pericardium 22 simultaneously. The adhesive is allowed to cure as previously described.

FIGS. 17 and 18 are perspective views of yet another embodiment of an adhesive grip 220 that is generally similar to the adhesive grip 150 shown in FIGS. 11-16. In this embodiment, the adhesive grip 220 includes a substantially hollow tubular body 221 provided with a plurality of cuts 224 at a distal end 222 to divide the distal end 222 into a plurality of flexible tabs 226. In FIG. 17 the tabs 226 are shown slightly flared or splayed radially outwardly from the axis of the grip 220. In reference to method 170 described above and shown in FIG. 12, during step 176, the grip 220 would be compressed against the pericardium 22, causing the tabs 226 to flare outward and flatten against the pericardium 22, as is shown in FIG. 18, and hence provide greater surface area for adhesive attachment. The tabs 226 are optionally provided by a flared silicon rubber material component. Optionally, the thickness of the tabs 226 is less than the thickness of the remainder of the grip 220. This encourages an inner surface of the tabs 226 to compress and flare radially outwardly against the pericardium 22. The tabs 226 may also be formed from a fabric material, for example, Dacron®. The adhesive grip 220 optionally has a coaxial construction, including an inner tubular member (not shown) as described with respect to the previous embodiment and shown in FIG. 16.

In any of the preceding embodiments described in relation to FIGS. 11-18, the installed adhesive grip may subsequently serve as a means of permanently or semi-permanently affixing an implanted medical device, such as a lead, to the pericardium 22. The lead is fixed to the installed adhesive grip by, for example, crimping the grip to the lead proximal to the surface of the pericardium 22. The adhesive bond may be reinforced with a suture to increase the fixation of the adhesive grip to the pericardium 22.

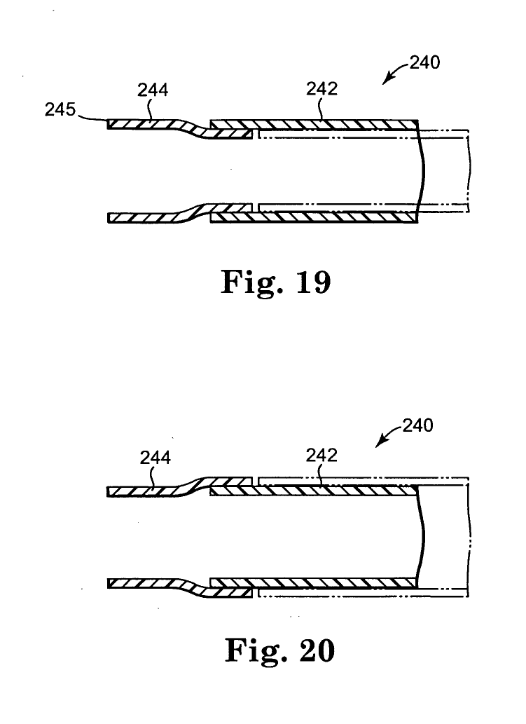

FIG. 19 shows another embodiment of an adhesive grip 240 that is generally similar to the adhesive grips shown in FIGS. 11-18. The adhesive grip 240 includes a substantially hollow tubular body 242 provided with a detachable distal tip 244 which may serve to affix an implantable medical device, such as a lead, to the pericardium 22. The detachable distal tip 244 includes a distal surface 245 which may be adhered to the pericardium 22 according to any of the embodiments as previously described. The lead is advanced through the tubular body 242 and affixed to the detachable tip 244 by, for example, crimping. The tubular body 242 is then detached from the tip 244 and removed.

In one embodiment, as is shown in FIG. 19, the detachable tip 244 is held deformably inside the tubular body 242. Once the detachable tip 244 is adhered to the pericardium 22, the longer tubular body 242 is pulled free of the now anchored tip 244. The detachable tip 244 may be forced from the remainder of the tubular body 242 by passing an inner semi-rigid tube (shown in dashed lines) through the tubular body 242 to push the tip 244 out of the end of the tubular body 242. According to another embodiment, the tip 244 is deformably held outside the end of the tubular body 242, as is shown in FIG. 20. Once the detachable tip 244 is adhered to the pericardium 22, the longer tubular body 242 is pulled free of the now anchored tip 244. An outer semi-rigid tube (shown in dashed lines) may be passed over the tubular body 242 to force the tip 244 from the end of the tubular body 242.

In addition to crimping, the tip 244 could be gently attached to the lead through the use of an internal lining of fabric, for example, Dacron®. The fit between the lead and the fabric lining would be sufficient to frictionally engage the lead to the tip 244. The fabric may be chosen to promote desired tissue ingrowth, thereby providing a more natural fixation of the tip 244 and lead at the tissue site.

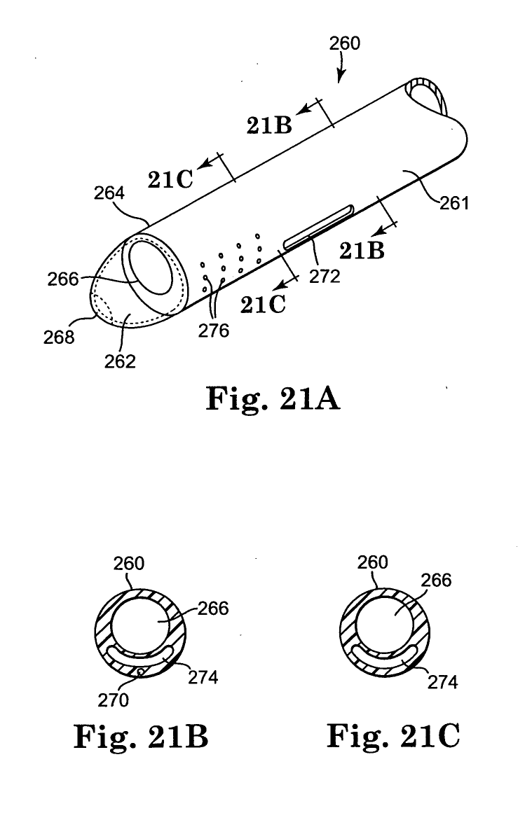

FIGS. 21A-21C show another embodiment of an adhesive grip 260 that is generally similar to the adhesive grips shown in FIGS. 11-18-20. As shown in FIG. 21A, adhesive grip 260 includes a tubular body 261 provided with an atraumatic tip 262 at a distal end 264. The tubular body 261 includes a main lumen 266 extending through a port 268 near the distal tip 262 and a secondary lumen 270 terminating in a secondary port 272 in a side wall of the tubular body 261 proximal to the distal tip 262 (compare FIGS. 21B and 21C). Main lumen 266 is adapted to slidably receive medical instruments, including guide wires, needles, forceps, endoscopes and the like. Secondary lumen 270 is adapted to slidably receive a perforation instrument as previously described. Grip 260 further includes an adhesive delivery lumen 274 terminating in a plurality of adhesive ports 276 in a side wall of the tubular body 261 proximal to the distal tip 262 and distal to the secondary port 272. Alternately, rather than a single adhesive delivery lumen 274 provided with a plurality of ports 276, grip 260 is provided with a plurality of adhesive delivery lumens each terminating in a single port positioned adjacent one another.

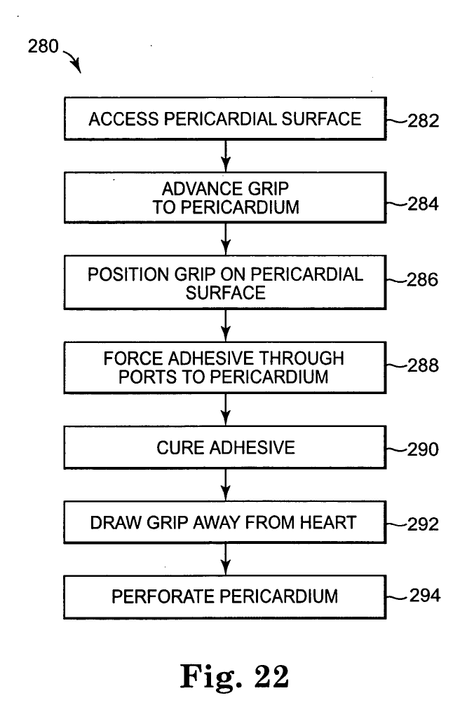

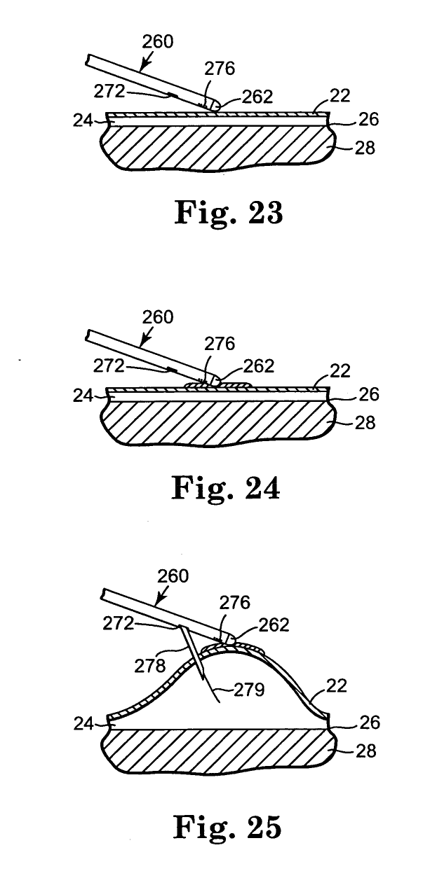

FIG. 22 shows a method 280 for accessing the pericardial space 24 using the adhesive grip 260 of FIGS. 21A-21C. In use a minimally-invasive surgical technique is used to gain access to a pericardium 22 (block 282). The grip is advanced to the pericardium 22 (block 284). The grip 260 is attached adhesively to the pericardium 22 as depicted in FIGS. 22 and 23. This includes the steps of positioning the grip on the pericardium 22 (block 286), forcing adhesive through ports 276 to the pericardium 22, as is shown in FIG. 23 (block 288) and curing the adhesive (block 290). The grip 260 is withdrawn, separating the pericardium 22 from the epicardium 26 and enlarging the pericardial space 24, as is shown in FIG. 25 (block 292). With the pericardium 22 withdrawn, a perforation implement is advanced through the secondary lumen 270 to perforate the pericardium 22, as is shown in FIG. 25 (block 294).

The grip 260 may be withdrawn axially from the surface of the pericardium 22, as is shown in FIG. 25. Alternately, the grip 260 is pivoted or rotated away from the pericardium 22 to drawn a portion of the pericardium 22 away from the epicardium 26. In such an embodiment, the perforation element can be angularly offset from the lumen 270 when advanced to perforate the pericardium 22.

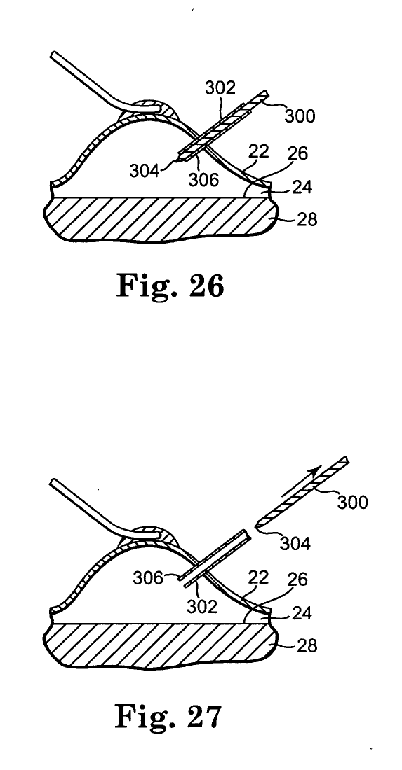

In the preceding embodiments, a hollow perforation instrument is used to perforate the raised pericardium 22, followed by insertion of an element such as a guide wire through the perforation instrument into the pericardial space. Other instruments and techniques may be used to perforate the raised pericardium 22. In one example, as is shown in FIGS. 26 and 27, a perforation instrument 300 is slidably disposed within an open sleeve 302 so that a distal pointed end 304 of the perforation instrument 300 protrudes from a distal end 306 of the sleeve 302. The perforation instrument 300 is used to perforate the raised pericardium 22 as previously described. The perforation instrument 300 is advanced until the distal end 306 of the sleeve 302 is located within the pericardial space 24. The perforation instrument 300 is then withdrawn while the sleeve 302 is held in place. Other payloads, for example, leads, may be then advanced through the sleeve 302 into the pericardial space 24.

Although the preceding embodiments are generally shown in relation to accessing the pericardial space 24, such methods and devices as described above may also be employed to access other tissue layers. For example, in the treatment of ischemias and aneurism, it may be desirable to access spaces between anatomic layers of the brain, including the dura. Such methods and devices may also be used to access the peritoneum. The methods and devices of the preceding embodiments may be used to access virtually any anatomic layer.

Various modifications and additions can be made to the exemplary embodiments discussed without departing from the scope of the present invention. Whereas the particular example of accessing the pericardial space has been used for illustrative purposes, it will be readily apparent to one skilled in the art that similar procedures in other anatomies may similarly benefit from the present invention. Accordingly, the scope of the present invention is intended to embrace all such alternatives, modifications, and variations as fall within the scope of the claims, together with all equivalents thereof.

We claim:

1. A method of accessing a pericardial space located between a pericardium and an epicardium, the method comprising:
attaching an adhesive grip to a surface of the pericardium;
grasping the adhesive grip;
drawing the adhesive grip away from the epicardium, thereby enlarging the pericardial space; and
perforating the pericardium with a perforation implement.

2. The method of claim 1 wherein the adhesive grip is an adhesive and further comprising curing the adhesive.

3. The method of claim 1 wherein the adhesive grip includes a gripping element and an adhesive.

4. The method of claim 3 wherein the gripping element includes a loop and attaching the gripping element with an adhesive further comprises:
applying the adhesive;
engaging the loop in the adhesive; and
curing the adhesive with the loop secured to the adhesive.

5. The method of claim 4 wherein the loop is coupled to an anchor.

6. The method of claim 3 wherein attaching the gripping element includes:
applying the adhesive to an end of a tubular body;
contacting the end of the tubular body to the pericardium; and
curing the adhesive.

7. The method of claim 6 wherein applying the adhesive includes injecting the adhesive through the tubular body.

8. The method of claim 6 wherein the gripping element includes a detachable tip coupled to the end of the tubular body and further comprising coupling a medical device to the detachable tip.

9. The method of claim 6 wherein contacting the end of the tubular body to the pericardium further comprises splaying a plurality of tabs at the end of the tubular body against the pericardium.

10. A method of accessing a pericardial space located between a pericardium and an epicardium, the method comprising:
attaching a first end of a gripping tube to a surface of the pericardium with an adhesive;
drawing the gripping tube away from the epicardium, thereby enlarging the pericardial space; and
perforating the pericardium with a perforation implement.

11. The method of claim 10 wherein attaching the first end of the gripping tube includes:
applying adhesive to at least one of the first end of the gripping tube and the surface of the pericardium;
contacting the first end of the gripping tube with the surface of the pericardium; and
curing the adhesive.

12. The method of claim 10 wherein attaching the first end of the gripping tube includes supplying adhesive through one or more adhesive lumens and ports.

13. The method of claim 10 wherein the first end of the gripping tube includes a detachable tip.

14. A pericardial access system for facilitating access to a pericardial space between a pericardium and an epicardium, the system comprising a tubular body defining an inner lumen adapted for slidably receiving a medical device therethrough and having a distal surface adapted for adhesively coupling to the pericardium, and a perforation instrument for perforating the pericardium, the instrument adapted to slide within the inner lumen and extend beyond the distal surface.

15. The system of claim 14 wherein a distal end of the tubular body is provided with at least one longitudinally extending cut defining a plurality of flexible tabs, the tabs having an inner facing surface that form the distal surface of the tubular body when splayed against the pericardium.

16. The system of claim 14 wherein the distal surface is located on a detachable distal tip coupled to a distal end of the tubular body.

17. The system of claim 14 further comprising a second tubular body slidably disposed within the inner lumen.

18. The system of claim 14 wherein the tubular body further comprises a second inner lumen adapted for receiving an adhesive, the second inner lumen terminating in at least a first port at the distal surface of the tubular body.

19. A pericardial grip that can be grasped for purposes of manipulating a pericardium, the pericardial grip comprising a graspable element and an adhesion means for facilitating adhesion of the graspable element to an exterior surface of the pericardium.

20. The pericardial grip of claim 19 wherein the graspable element is a loop of suture material.

21. The pericardial grip of claim 19 wherein the graspable element is a tubular body having a distal surface adapted for adhering to the surface of the pericardium.

22. The pericardial grip of claim 21 wherein the adhesion means includes a plurality of flexible tabs located at a distal end of the tubular body adapted for adhering to the pericardium.

23. The pericardial grip of claim 21 wherein the adhesion means comprises a bolus of an adhesive material deposited on a surface of the pericardium.

24. The pericardial grip of claim 23 wherein the adhesive is selected from the group consisting of: BioGlue®, fibrin sealants, cyanoacrylates, bovine collagen/thrombin, polyethylene glycol polymers and Loctite® acrylic adhesive.
